(19) United States
(12) Patent Application Publication
(10) Pub. No.: US 2007/0248995 A1
(43) Pub. Date: Oct. 25, 2007

(54) Molecules

(76) Inventors: Tomas Drmota, Molndal (SE); Susanna Engberg, Molndal (SE); Bengt Von Mentzer, Molndal (SE)

Correspondence Address: WHITE & CASE LLP;PATENT DEPARTMENT, 1155 AVENUE OF THE AMERICAS, NEW YORK, NY, 10036, US

(21) Appl. No.: 10/592,929

(22) PCT Filed: Mar. 16, 2005
(86) PCT No.: PCT/GB05/01008

§ 371 Date: Sep. 15, 2006

(30) Foreign Application Priority Data
Mar. 20, 2004 (GB) 0406342.6

(51) Int. Cl. G01N 33/53; A61K 38/00; C07K 14/00; C07K 14/705; C12N 15/00; C12P 21/04

(52) U.S. Cl. 435/7.8; 435/320.1; 435/325; 435/69.1; 514/2; 530/350; 536/23.1

(57) ABSTRACT

This invention relates to the gene encoding gerbil neurokinin 1 receptor (gNK1r) and to proteins encoded by the gene, as well as to means of regulating their biological activity. In addition the invention relates to the use of the gene and protein to identify therapeutic agents for controlling asthma, pain, depression, anxiety, emesis, inflammatory bowel disease (IBD), irritable bowel syndrome (IBS), functional dyspepsia, migraine, urinary incontinence and schizophrenia.

This application is the U.S. National Stage of International Application No. PCT/GB2005/001008, filed Mar. 16, 2005.

This invention relates to the gene encoding gerbil neurokinin 1 receptor (gNK1r). The invention further relates to proteins encoded by the gene and to means of regulating their biological activity. In addition the invention relates to the use of the gene and protein to identify therapeutic agents for controlling asthma, pain, depression, anxiety, emesis, inflammatory bowel disease (IBD), irritable bowel syndrome (IBS), functional dyspepsia, migraine, urinary incontinence and schizophrenia.

Neurokinin receptors (NK) are class I (A) family G-protein coupled receptors (GPCR's). They are linked to at least two G-proteins. All NK receptors couple to Gq which leads to mobilization of intracellular calcium. However, NK1r has the additional capability of coupling to Gs, which in turn leads to activation of cAMP production. The endogenous ligands for all three receptors (NK1r, NK2r and NK3r) act as full agonists on their preferred receptor. Substance P has the highest affinity for NK1r, Neurokinin A for the NK2r and Neurokinin B for the NK3r; however all tachykinins can interact with each receptor.

The NK receptors have been considered as attractive targets in a wide variety of indications such as asthma, pain, depression, anxiety, emesis, inflammatory bowel disease (IBD), irritable bowel syndrome (IBS), functional dyspepsia, migraine, urinary incontinence and schizophrenia. Compounds antagonizing NK1r have received the greatest amount of attention, although in recent years the interest for both NK2r and NK3r antagonists has increased. In addition, nonselective compounds with dual activity have also been explored. Despite the large interest, several clinical trials have had disappointing outcomes with various NK1r antagonists, especially within the pain and asthma areas. In 2003, the first NK1r antagonist, aprepitant or MK869, reached the market. The compound is approved for the treatment of emesis during cytostatic therapy.

During the development of NKr antagonists it was realised that most compounds with high affinity for human NK1r had low affinity for rat and mouse NK1r . These species differences in compound affinity were most evident for NK1r, but species differences exist for NK2r and NK3r albeit to a lesser degree. The cloned rat and human NK1r show about 95% identity. However, when analyzing the seven transmembrane regions, six amino acids differ between the two species. The critical amino acids for antagonist binding are believed to be those at positions 116 and 290 and these differ in the rat and human.

Several non-rat/mouse in vivo models have been developed to screen for efficacy of NK1r antagonists in vivo. A few of the indications in which NK1r antagonists have attracted attention require central exposure of the compound (i.e. anxiety, depression, pain). One of these models is the gerbil foot-tapping model (Bristow and Young. Eur J Pharmacol 253:245-252, 1994). This model is induced by the central (intracerebroventricular, i.c.v.) administration of selective NK1r agonists. This treatment results in repetitive hind-feet tapping of the gerbil, which can easily be recorded. Pre-treatment with potent and CNS-penetrable NK1r antagonists will thus inhibit foot tapping induced by agonist. This model has been very useful in screening for antagonists with central activity (Megens et al, JPET, 302:696-709, 2002; Rupniak et al, Neuropharmacology 45:231-241, 2003).

Although the NK1r gene has been cloned from some species, the gerbil sequence has to date not been cloned. The inventors have surprisingly found that in vitro recombinant screens utilizing the gerbil NK1r or the human NK1r give similar results to each other, whereas the rat NK1r gives more dissimilar results. This makes the in vitro screen using the gerbil NK1r sequence more human-like than that of the rat, making the gerbil sequence the preferred non-human sequence. The value of using a non-human sequence that is human-like in identifying potential modulatory test compounds for treating humans arises because these test compounds will have to be tested in animal in vivo models. Thus, if a test compound looks promising in the gerbil in vitro screen and the human in vitro screen and the gerbil in vivo model, there is greater confidence in its value in treating humans, and thus, is more likely to be approved for testing in humans.

The present invention discloses full-length cDNA and protein sequences for the gerbil NK1r protein (gNK1r). gNK1r protein shares 94.9% sequence identity with human NK1r sequence and 96.9% sequence identity with rat NK1r sequence. Importantly however, at the three crucial amino acids that form part of the antagonist binding site (positions 80, 116 and 290), the gerbil and human proteins possess the identical residue, whereas in the rat there are different amino acid residues at these critical positions (see Table 3).

The invention further discloses that gNK1r has utility in in vitro screens for compounds that modulate NK1r. Such compounds may have utility as therapeutics in treating diseases or conditions such as asthma, pain, depression, anxiety, emesis, inflammatory bowel disease (IBD), irritable bowel syndrome (IBS), functional dyspepsia, migraine, urinary incontinence and schizophrenia.

Unless defined otherwise, all technical and scientific terms used herein have the same meaning as is commonly understood by one of skill in the art to which this invention belongs. All publications and patents referred to herein are incorporated by reference.

In a first aspect of the present invention we provide an isolated polynucleotide molecule comprising a nucleic acid sequence which encodes a gNK1r polypeptide. By the term “isolated”, we mean that the polynucleotide molecule has been separated from those constituents that are normally present with it in nature. Preferably the gNK1r polypeptide has the sequence depicted in SEQ ID NO: 2. An isolated gNK1r may or may not be membrane bound.

The invention includes sequences at least 97% identical (preferably at least 98% identical, more preferably at least 99% identical) to the sequences of the invention as determined by the Smith-Waterman algorithm.

In another aspect of the present invention we provide an isolated polynucleotide molecule comprising a nucleic acid sequence which encodes a polypeptide having at least 98% sequence identity to the amino acid sequence disclosed in SEQ ID NO: 2. Isolated polynucleotides of the present invention include sequences which comprise the gNK1r cDNA sequence set out in SEQ ID NO: 1.

In a further aspect of the invention we provide C-terminal and N-terminal fragments of the isolated polynucleotide or polypeptide molecules of the present invention. Preferred polypeptide fragments are at least 350 amino acids in length, preferably at least 377 amino acids in length, and more preferably at least 387 amino acids in length. Indeed any convenient fragment of the polynucleotide molecule may be a useful fragment for further research, therapeutic or diagnostic purposes. Further convenient fragments include those whose termini are defined by restriction sites within the molecule of one or more kinds, such as any combination of Rsa1, Alu1 and Hinf1.

In a further aspect we provide an expression vector comprising a polynucleotide molecule of the present invention.

A variety of mammalian expression vectors may be used to express the recombinant polypeptides of the present invention. Commercially available mammalian expression vectors which are suitable for recombinant expression include, pcDNA3 (Invitrogen), pMC1neo (Stratagene), pXT1 (Stratagene), pSG5 (Stratagene), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146), pUCTag (ATCC 37460), 1ZD35 (ATCC 37565), pLXIN, pSIR (CLONTECH), and pIRES-EGFP (CLONTECH).

Baculoviral expression systems may also be used with the present invention to produce high yields of biologically active polypeptides. Preferred vectors include the CLONTECH, BacPak™ Baculovirus expression system and protocols which are commercially available (CLONTECH, Palo Alto, Calif.).

Further preferred vectors include vectors for use with the mouse erythroleukaemia cell (MEL cell) expression system comprising the human beta globin gene locus control region (Davies et al., J. of Pharmacol. and Toxicol. Methods 33(3):153-158, 1995).

Vectors comprising one or more polynucleotide molecules of the present invention may then be purified and introduced into appropriate host cells. Therefore in a further aspect we provide a transformed host cell comprising a polynucleotide molecule of the present invention.

The polypeptides of the present invention may be expressed in a variety of hosts such as bacteria, plant cells, insect cells, fungal cells and human and animal cells. Eukaryotic recombinant host cells are especially preferred. Examples include yeast, mammalian cells including cell lines of human, bovine, porcine, monkey and rodent origin, and insect cells including Drosophila and silkworm-derived cell lines. Cell lines derived from mammalian species which may be used and which are commercially available include, L cells L-M(TK—) (ATCC CCL 1.3), L cells L-M (ATCC CCL 1.2), HEK 293 (ATCC CRL 1573), Raji (ATCC CCL 86), CV-1 (ATCC CCL 70), COS-1 (ATCC CRL 1650), COS-7 (ATCC CRL 1651), CHO—K1 (ATCC CCL 61), 3T3 (ATCC CCL 92), NIH/3T3 (ATCC CRL 1658), HeLa (ATCC CCL 2), C127I (ATCC CRL 1616), BS—C-1 (ATCC CCL 26) and MRC-5 (ATCC CCL 171).

The expression vector may be introduced into host cells to express a polypeptide of the present invention via any one of a number of techniques including calcium phosphate transformation, DEAE-dextran transformation, cationic lipid mediated lipofection, electroporation or infection

The transformed host cells are propagated and cloned, for example by limiting dilution, and analysed to determine the expression level of recombinant polypeptide. Identification of transformed host cells which express a polypeptide of the present invention may be achieved by several means including immunological reactivity with antibodies described herein and/or the detection of biological activity.

Polypeptides of the present invention may be expressed as fusion proteins, for example with one or more additional polypeptide domains added to facilitate protein purification. Examples of such additional polypeptides include metal chelating peptides such as histidine-tryptophan modules that allow purification on immobilised metals (Porath, J., Protein Exp. Purif. 3:263 (1992)), protein A domains that allow purification on immobilised immunoglobulin, and the domain utilised in the FLAGS extension/affinity purification system (Immunex Corp, Seattle Wash.). The inclusion of cleavable linker sequences such as Factor XA or enterokinase (Invitrogen, San Diego Calif.) between the purification domain and the coding region is useful to facilitate purification. A preferred protein purification system is the CLONTECH, TALON™ nondenaturing protein purification kit for purifying 6xHis-tagged proteins under native conditions (CLONTECH, Palo Alto, Calif.).

Therefore in a further aspect we provide a method for producing a polypeptide of the present invention, which method comprises culturing a transformed host cell comprising a polynucleotide of the present invention under conditions suitable for the expression of said polypeptide.

In another aspect of the present invention we provide an isolated or purified gNK1receptor polypeptide or a polypeptide fragment thereof of greater than 350 amino acids. Preferably the gNK1r polypeptide or fragment thereof is selected from:

i) SEQ ID NO: 2 or a fragment thereof selected from SEQ ID NO: 2 positions 1-377, 1-387, 1400, 1-406, 10-407, 15-407, 20-407, 25-407, 35-407, 40-390, 15-385, 20-380, 25-370, 30-370 and 25-400.

In a further aspect of the present invention we provide a purified polypeptide comprising the gNK1r amino acid sequence set out in SEQ ID NO: 2 or a variant of SEQ ID NO: 2 having at least 98% identity thereto, or an N-terminal or C-terminal fragment thereof of at least 387 amino acids in length.

A variant is a polynucleotide or polypeptide which differs from a reference polynucleotide or polypeptide, but which retains some of its essential characteristics. For example, a variant of a gNK1r polypeptide may have an amino acid sequence that is different by one or more amino acid substitutions, deletions and/or additions. The variant may have conservative changes (amino acid similarity), wherein a substituted amino acid has similar structural or chemical properties, for example, the replacement of leucine with isoleucine. Alternatively, a variant may have non-conservative changes, e.g., replacement of a glycine with a tryptophan. Guidance in determining which and how many amino acid residues may be substituted, inserted or deleted and the effect this will have on biological activity may be reasonably inferred from the present disclosure by a person skilled in the art and may further be found using computer programs well known in the art, for example, DNAStar software.

Amino acid substitutions may be made, for instance, on the basis of similarity in polarity, charge, solubility, hydrophobicity, hydrophilicity, and/or the amphipathic nature of the residues. Negatively charged amino acids, for example, include aspartic acid and glutamic acid; positively charged amino acids include lysine and arginine; and amino acids with uncharged polar head groups having similar hydrophilicity values include leucine, isoleucine, valine, glycine, alanine; asparagine, glutamine; serine, threonine, phenylalanine, and tyrosine.

Suitable substitutions of amino acids include the use of a chemically derivatised residue in place of a non-derivatised residue. D-isomers and other known derivatives may also be substituted for the naturally occurring amino acids. See, e.g., U.S. Pat. No. 5,652,369, Amino Acid Derivatives, issued Jul. 29, 1997. Example substitutions are set forth in Table 1.

“Homology” as used in this description is a measure of the similarity or identity of nucleotide sequences or amino acid sequences. In order to characterise the homology, subject sequences are aligned so that the highest order identity match is obtained. Identity can be calculated using published techniques. Computer program methods to determine identity between two sequences, for example, include DNAStar software (DNAStar Inc., Madison, Wis.); the GCG program package (Devereux, J., et al., Nucleic Acids Research 1984, 12(1):387); and BLASTP, BLASTN, FASTA (Atschul, S. F. et al., J Molec Biol 1990, 215:403). Sequence identity can be determined conventionally using the well known computer program, BESTFIT (Wisconsin Sequence Analysis Package, Version 8 for Unix, Genetics Computer Group, University Research Park, 575 Science Drive, Madison, Wis., 53711). When using BESTFIT or another sequence alignment program to determine the similarity of a particular sequence to a reference sequence, the parameters are typically set such that the percentage identity is calculated over the full length of the reference nucleotide sequence or amino acid sequence and that gaps in homology of up to about 10% of the total number of nucleotides or amino acid residues in the reference sequence are allowed. The above-identified software packages endeavour to closely approximate the “gold-standard” alignment algorithm of Smith-Waterman. Thus, the preferred software/search engine programme for use in assessing the percent identity, i.e how two primary polypeptide sequences line up is Smith-Waterman.

In a further aspect we provide natural allelic polymorphic variants of the polynucleotides and polypeptides of the present invention. Polymorphisms are variations in polynucleotide or polypeptide sequences between one individual and another. DNA polymorphisms may lead to variations in amino acid sequence and consequently to altered protein structure and functional activity. Polymorphisms may also affect mRNA synthesis, maturation, transport and stability. Polymorphisms which do not result in amino acid changes (silent polymorphisms) or which do not alter any known consensus sequences may nevertheless have a biological effect, for example by altering mRNA folding or stability.

The Applicants have surprisingly found that the amino acid residues at position 80, 116 and 290 of gNK1r are the same as in human NK1r. These residues are believed to be critical for antagonist binding, which may explain why the gerbil receptor gives more comparable results to the human receptor than the rat receptor does to the human receptor.

In one embodiment of the invention, the variant gNK1r polypeptide possesses, relative to the position in SEQ ID NO: 2, a serine at position 80, a valine at position 116 and an isoleucine at position 290.

In a further aspect of the invention we provide a method for identifying a chemical compound capable of modulating the activity of gNK1r which method comprises:
(i) contacting a chemical compound with an gNK1r polypeptide of the invention described herein; and
(ii) measuring an effect of the chemical compound on the activity of the gNK1r.

In a further aspect of the invention we provide a method for identifying a therapeutic agent capable of modulating the activity of gNK1r, which method comprises:
(i) contacting a candidate compound modulator with an gNK1r polypeptide comprising the amino acid sequence set out in SEQ ID NO: 2 or a variant of SEQ ID NO: 2 having at least 98% sequence identity thereto; and
(ii) measuring an effect of the candidate compound modulator on the activity of the gNK1r polypeptide.

In embodiments of the above two methods, the gNK1r polypeptide can be membrane bound.

In a further embodiment, after the gNK1r in vitro screening methods described herein an identified gNK1r modulatory test compound is subsequently tested in an in vivo animal model, such as the gerbil foot tap model.

Thus, according to another aspect of the invention there is provided a method for screening compounds for their potential as human therapeutic NK1r modulatory compounds comprising:
(i) identifying putative NK1r modulatory compounds according to their ability to modulate human NK1r or gerbil NK1r in am in vitro assay; and
(ii) testing a compound that demonstrates modulatory activity against human NK1r or gerbil NK1r in (i) above, in a gerbil in vivo model of NK1r activity,

wherein a compound that modulates NK1r in (i) and (ii) above, is one that has potential as a human therapeutic NK1r modulatory compound.

According to another aspect of the invention there is provided a method for screening compounds for their potential as human therapeutic NK1r modulatory compounds comprising:
(iii) identifying putative NK1r modulatory compounds according to their ability to modulate human NK1r and gerbil NK1r in in vitro assays; and
(iv) testing a compound that demonstrates modulatory activity against human NK1r and gerbil NK1r in (i) above, in a gerbil in vivo model of NK1r activity,
   wherein a compound that modulates NK1r in (i) and (ii) above, is one that has potential as a human therapeutic NK1r modulatory compound.

In separate embodiments of the two aspects immediately above, the in vitro assays of (i) can be carried out separately or concurrently.

In another embodiment, the gerbil in vivo model of NK1r activity is the gerbil foot tap model.

In another embodiment, the in vitro assays used to measure the ability of the test compound to modulate human and/or gerbil NK1r utilises recombinant human NK1r and/or gerbil NK1r, as appropriate. In another embodiment, the in vitro assay used to measure the ability of a test compound to modulate gerbil NK1r is any of the methods disclosed herein.

Modulation of the activity of gNK1r comprises either stimulation or inhibition. Thus a therapeutic agent capable of modulating the activity of gNK1r is an agent that either stimulates or inhibits the activity of gNK1r. The terms “modulator of gNK1r activity” and “gNK1r modulator” are also used herein to refer to an agent that either stimulates or inhibits the activity of gNK1r. The therapeutic agents of the invention have utility in treating or controlling asthma, pain, depression, anxiety, emesis, inflammatory bowel disease (IBD), irritable bowel syndrome (IBS), functional dyspepsia, migraine, urinary incontinence or schizophrenia.

In a further aspect of the invention we provide a screen for identifying compounds which modulate the activity of gNK1r, the invention extends to such a screen and to the use of compounds obtainable therefrom to modulate the activity of gNK1r in vivo.

Potential therapeutic agents which may be tested in the screen include simple organic molecules, commonly known as “small molecules”, for example those having a molecular weight of less than 2000 Daltons. The screen may also be used to screen compound libraries such as peptide libraries, including synthetic peptide libraries and peptide phage libraries. Other suitable molecules include antibodies, nucleotide sequences and any other molecules which modulate the activity of gNK1r.

Once an inhibitor or stimulator of gNK1r activity is identified then medicinal chemistry techniques can be applied to further refine its properties, for example to enhance efficacy and/or reduce side effects.

It will be appreciated that there are many screening procedures which may be employed to perform the present invention. Examples of suitable screening procedures which may be used to identify a gNK1r modulator for use in controlling or treating a disease selected form the group consisting of: asthma, pain, depression, anxiety, emesis, inflammatory bowel disease (IBD), irritable bowel syndrome (IBS), functional dyspepsia, migraine, urinary incontinence and schizophrenia, include rapid filtration of equilibrium binding mixtures, enzyme linked immunosorbent assays (ELISA), radioimmunoassays (RIA) and fluorescence resonance energy transfer assays (FRET). For further information on FRET the reader is directed to International Patent Application WO 94/28166 (Zeneca). Methods to identify potential drug candidates have been reviewed by Bevan P et al., 1995, TIBTECH 13 115.

A preferred method for identifying a compound capable of modulating the activity of gNK1r is a scintillation proximity assay (SPA). SPA involves the use of fluomicrospheres coated with acceptor molecules, such as receptors to which a ligand will bind selectively in a reversible manner (N Bosworth & P Towers, Nature, 341, 167-168, 1989). The technique requires the use of a ligand labelled with an isotope that emits low energy radiation which is dissipated easily into an aqueous medium. At any point during an assay, bound labelled ligands will be in close proximity to the fluomicrospheres, allowing the emitted energy to activate the fluor and produce light. In contrast, the vast majority of unbound labelled ligands will be too far from the fluomicrospheres to enable the transfer of energy. Bound ligands produce light but free ligands do not, allowing the extent of ligand binding to be measured without the need to separate bound and free ligand.

Cellular assay systems may be used to further identify gNK1r modulators for use in treating any of the diseases identified above.

Therefore in a further aspect of the invention we provide a method for identifying a therapeutic agent capable of modulating the activity of gNK1r, which method comprises:
(i) contacting a candidate compound modulator with a host cell which expresses a gNK1r polypeptide comprising the amino acid sequence set out in SEQ ID NO: 2 or a variant of SEQ ID NO: 2 having at least 98% identity thereto, or a biologically active fragment thereof; and
(ii) measuring an effect of the candidate compound modulator on the activity of gNK1r.

A preferred cellular assay system for use in the method of the invention is a two-hybrid assay system. The two-hybrid system utilises the ability of a pair of interacting, proteins to bring the activation domain of a transcription factor into close proximity with its DNA-binding domain, restoring the functional activity of the transcription factor and inducing the expression of a reporter gene (S Fields & O Song, Nature, 340, 245-246, 1989). Commercially available systems such as the Clontech Matchmaker™ systems and protocols may be used with the present invention.

Other preferred cellular assay systems include measurement of changes in the levels of intracellular signalling molecules such as cyclic-AMP, intracellular calcium ions, or arachidonic acid metabolite release. These may all be measured using standard published procedures and commercially available reagents. In addition the polynucleotides of the present invention may be transfected into appropriate cell lines that have been transfected with a “reporter” gene such as bacterial lacZ, luciferase, aequorin or green fluorescent protein that will “report” these intracellular changes (Egerton et al, J. Mol, Endocrinol, 1995, 14(2), 179-189).

According to a further aspect of the invention we provide a method of making a pharmaceutical composition, which comprises:
(i) identifying a chemical compound capable of modulating the activity of gNK1r; and
(ii) mixing the compound thus identified with a pharmaceutically acceptable diluent or carrier.

According to a further aspect of the invention, we provide a pharmaceutical composition which comprises a gNK1r modulator, or a pharmaceutically acceptable salt thereof, in association with a pharmaceutically acceptable diluent or carrier.

The composition may be in the form suitable for oral use, for example a tablet, capsule, aqueous or oily solution, suspension or emulsion; for topical use, for example a cream, ointment, gel or an aqueous or oily solution or suspension; for nasal use, for example a snuff, nasal spray or nasal drops; for rectal use, for example a suppository; for administration by inhalation, for example as a finely divided powder such as a dry powder, a microcrystalline form or a liquid aerosol; for sub-lingual or buccal use, for example a tablet or capsule; or for parenteral use (including intravenous, subcutaneous, intramuscular, intravascular or infusion), for example a sterile aqueous or oily solution or suspension. In general, the above compositions may be prepared in a conventional manner using conventional excipients.

The invention also provides the use of a gNK1r modulator in the production of a medicament for use in the treatment of a disease in humans selected from: asthma, pain, depression, anxiety, emesis, inflammatory bowel disease (IBD), irritable bowel syndrome (IBS), functional dyspepsia, migraine, urinary incontinence and schizophrenia.

The amount of active ingredient that is combined with one or more excipients to produce a single dosage form will necessarily vary depending on the subject treated and the particular route of administration. For example, a formulation intended for oral administration to humans will generally contain for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient.

The size of the dose for therapeutic or prophylactic purposes of a gNK1r modulator will naturally vary according to the nature and severity of the disease, the age and sex of the patient, and the route of administration, according to well known principles of medicine.

In using a gNK1r modulator for therapeutic or prophylactic purposes it will generally be administered so that a daily dose in the range for example 0.5 mg to 75 mg per kg body weight is received, given if required in divided doses. In general lower doses will be administered when a parenteral route is employed. Thus for example, for intravenous administration, a dose in the range for example 0.5 mg to 30 mg per kg body weight will generally be used. Similarly, for administration by inhalation a dose in the range for example 0.5 mg to 25 mg per kg body weight will be used.

The invention will now be illustrated but not limited by reference to the following Tables, Examples and Figures. Unless indicated otherwise, the techniques used are those detailed in well known molecular biology textbooks such as Sambrook, Fritsch & Maniatis, Molecular Cloning a Laboratory Manual, second edition, 1989, Cold Spring Harbor Laboratory Press.

### FIGURE LEGENDS

FIG. 1 Shows the alignment of human, gerbil and rat NK1 receptor proteins. The differences are highlighted in bold.

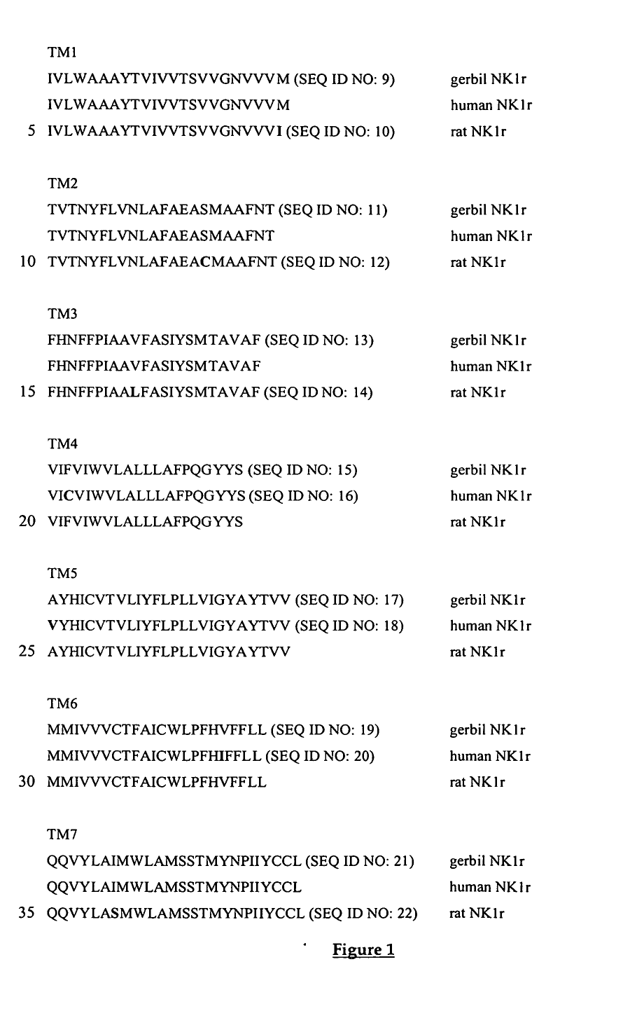

FIG. 2 Shows the effect of ASMSP and GR73632 on GFT. The effect is expressed as rate of tapping (rate/minute). The submaximal effective dose was 10 pmol/gerbil which was also the dose used for estimation of the inhibitory potency of NK receptor antagonists (FIG. 3)

FIG. 3 Histograms showing the inhibitory effect of NK1 receptor antagonists ZD 6021 (A), CP 122,721 (B), MK 869 (C), and CP 99,994 (D) on GFT. The IC₅₀ value of each compound is shown under each figure. The IC₅₀ values are determined as inhibition of ASMSP(10 nM)-induced foot tapping. The dose used represented a submaximal (80%) agonist concentration.

### EXAMPLE 1

Experimental Procedure of Gerbil Molecular Cloning

Primer Selection

The primers for use in cloning the gerbil NK1r had to be predicted based on likely conserved regions identified by alignment of the human, rat and mouse sequences. Numerous primers so designed were unsuitable for PCR cloning of the gNK1r and had to be redesigned to optimise the annealing temperature for the gerbil sequence. Eventually, six primers were chosen for NK1r cloning (see Table 1). All primers used for PCR were ordered from Qiagen (http://www.oligos.qiagen.com).

TABLE 1

Primers used for cloning of human and
gerbil NK1r. These primers represent
SEQ ID Nos: 3-8.

Genbank
Accession

| | |
|---|---|
| Primer name and sequence | No. (Sequence |

| | | |
|---|---|---|
| (5′-3′) | position) | Species |

| | | |
|---|---|---|
| # NK1-10; | S62045, x62932, | /human/ |
| GCTGCCCTTCCACATCTTCTTCCTCC | X64323 | mouse/ |
| TG | (783-810) | guinea |
| (SEQ ID NO: 3) | | pig |
| # NK1-12; | S62045, J05097, | human/ |
| GCCAGCAGGAGAGCCAGGACCCAGAT | x62932 | rat/ |
| G | (485-459) | mouse |
| (SEQ ID NO: 4) | | |
| # NK1-16; | | gerbil |
| AGGCATCTGCAACAAGGTC | | |
| (SEQ ID NO: 5) | | |
| # NK1-17; | | gerbil |
| AACCATTATGACCCTTTCCAGA | | |
| (SEQ ID NO: 6) | | |
| # NK1-18; | | gerbil |
| GGATCCGCCACCATGGATAACGTCCT | | |
| CCCTGG | | |
| (SEQ ID NO: 7) | | |
| # NK1-19; | | gerbil |
| GATATCATGCCCTTGAAATATGCCCA | | |
| CTG | | |
| (SEQ ID NO: 8) | | |

TABLE 2
Sequence homology of NK1 receptors. The gerbil NK1 receptor
sequence is compared with human and rat.

| | Human NK1 | Rat NK1 |
|---|---|---|
| Gerbil NK1 | 94.9% | 96.9% |

TABLE 3
Comparison of sequence homology in transmembrane regions.

| | Human NK1 | Rat NK1 |
|---|---|---|
| Gerbil NK1 TM1 | 100% | 95.7% |
| Gerbil NK1 TM2 | 100% | 95.5% |
| Gerbil NK1 TM3 | 100% | 95.5% |
| Gerbil NK1 TM4 | 95% | 100% |
| Gerbil NK1 TM5 | 96% | 100% |
| Gerbil NK1 TM6 | 95.5% | 100% |
| Gerbil NK1 TM7 | 100% | 96% |

Interestingly, the gerbil and human sequences show complete identity for transmembrane regions 2, 3 and 7, the crucial regions for antagonist binding.
Total RNA Extraction

Total RNA was prepared from 20-100 mg of gerbil tissue with RNA-STAT-60 (Tel Test Inc) according to the manufacturer's instructions. RNA was quantified and used in first strand cDNA synthesis by absorbance measurements at 260 mn.

Molecular Cloning of Nk1 Receptors

RACE gNK1r:

In the first strand synthesis, 1 μg of total RNA from striatum and pancreas respectively were used. The reactions were performed using SMART RACE cDNA Amplification kit (BD Biosciences)

To generate species-specific sequence for gerbil NK1r, SMART RACE cDNA Amplification kit (BD Biosciences) was used. ClusalW alignment of NK1r sequence from human, rat, mouse and guinea pig was used to select primers with high homology between different species. Both 5′RACE and 3′RACE were preformed using the SMART RACE cDNA kit. Primer #NK1-10 was used in the 3′RACE and #NK1-12 was used in the 5′RACE. The RACE reactions were performed on cDNA from gerbil pancreas. The RACE fragments were characterized and two clones contained gerbil-specific NK1r in the untranslated region. Gerbil-specific primers were constructed (#NK1-16 and #NK1-17).

PCR gNK1r:

Full-length PCR was performed using primers #NK1-16 and #NK1-17. Complementary DNA from striatum (2.5 μl) was used in the full-length PCR with forward and reversed primer (1×PCR Buffer, 5 mM of each dNTP, 20 μM primers and 1 U Pfu Ultra (Stratagene)). The PCR conditions used were: 1 cycle at 95° C. for 2 min; 35 cycles at 95° C. denaturation for 30 sec, 55° C. annealing for 1 min, 72° C. extension for 2 min; I cycle at 72° C. final extension for 10 min. The resulting PCR product was cloned into pCR®4Blunt-TOPO vector (Invitrogen) and 10 different clones were sequenced. One of the clones was then used in a second PCR with primer #NK1-18 and primer #NK1-19 containing restrictions site for sub cloning in the pIREShyg2 expression vector.

Cloning of Human NK1 Receptor

Cloning of human NK1r was carried out as outlined in Caccesse et al. (Neuropeptides 33: 239-243,1999) and stably transfected into CHO cells as below.

Cloning of Rat NK1 Receptor

Rat NK1r was cloned by PCR from rat brain cDNA library, using the high fidelity DNA polymerase, pfu (Stratgene), and the primers designed were based on the DNA sequence of rat Substance P receptor (SPR) in the GenBank Accession No. J05097. The PCR product was 1249 bp in length, being 1224 bp of the gene and additional terminal cloning site sequence, an extra, “stop” codon and inclusion of the Kozak sequence (GCCACC). The PCR product was cloned into the pcDNA 3.1 Hygro vector at the Not I site and then transformed into XL10 Gold cells (Stratgene). Correct orientation was confirmed and the selected clone was fully sequenced.

Stable Transfection

Gerbil and human NK1r DNA prepared above, was transfected into CHO cells grown in DMEM/F-12 (1:1) with GlutaMax I (Gibco/BRL #31331-028) supplemented with 10% FBS (Gibco/BRL) #10270-106) using Lipofectamine™ 2000 according to the manufacturer's protocol (Invitrogen). To transfect 3.5×10⁶ cells, 8 μg DNA and 30 μl of LF 2000 were used. The cells were cultured without selection for 24 hours. To achieve selection for hygromycin-resistant clones 500 μg/ml hygromycin was added to the medium.

The CHO cells stably transfected with NK1r were cultured in a humidified incubator under 5% CO₂, in Nut Mix F12 (HAM) with Glutamax I, 10% Foetal Bovine Serum (FBS), 1% Penicillin/Streptomycin (PEST) supplemented with 200 μg/ml Geneticin for the gNK1R and hNK2R expressing cells. The cells were grown in T175 flasks and routinely passaged when 70-80% confluent for up to 20-25 passages.

Rat NK1r DNA prepared above was stably transfected using a routine Lipofectamine method. Cells were washed (PBS w/o Ca2+, Mg2+) with 5 ml Opti-Mem and thereafter incubated in 5 mi Opti-Mem for approximately one hour. To a clear plastic tube was added 2-3 μg of DNA construct and 4 ml Opti-Mem and gently agitated. The reaction mixture was incubated for 45 minutes at room temperature. Before adding the transfection mixture to the cells, the cell medium was removed and transfection mixture was added and incubated at 37° C. for 5 hours. The transfection medium was then removed and 14 ml of cultivation medium (Nut Mix F12, Ham's supplemented with 10% FBS and 1% PEST) without selection factors was added. The cells were then incubated for a further 24 hours. The cells were then washed with 5 ml PBS and detached with 6 ml PBS with trypsin (PBS w/o Ca2+, Mg2+ Trypsin/EDTA), and then routinely passaged.

Activation of NK1 Receptors

The potency of different agonists to activate NK1r measured as NKr-mediated increase in intracellular Ca²⁺ was assessed by the following procedure:

CHO cells stably transfected with NK1r were plated in black-walled/clear-bottomed 96-well plates (Costar 3904) at 3.5×10⁴ cells per well and grown for approximately 24 hours in normal growth media in a 37° C. CO₂-incubator.

Before the FLIPR assay, the cells of each 96-well plate were loaded with the Ca²⁺-sensitive dye Fluo-3 (TEFLABS 0116) at 4 μM in a loading media consisting of Nut Mix F12 (HAM) with Glutamax I, 22 mM HEPES, 2.5 mM Probenicid (Sigma P-8761) and 0.04% Pluronic F-127 (Sigma P-2443) and kept in the dark for 1 hour in a 37° C. CO₂-incubator. The cells were then washed three times in assay buffer (Hanks balanced salt solution (HBSS) containing 20 mM HEPES, 2.5 mM Probenicid and 0.1% BSA) using a multi-channel pipette leaving them in 150 μl at the end of the last wash. Serial dilutions of a test agonist in assay buffer were automatically pipetted by FLIPR (Fluorometric Imaging Plate Reader) into each test well and the fluorescence intensity was recorded (excitation 488 nm and emission 530 nm) by the FLIPR CCD camera for approximately 2 minutes. The response was measured as the peak relative fluorescence after agonist addition and EC₅₀s were calculated from ten-point concentration-response curves for each agonist using X1fit (model 201).

Inhibition of NK1 Receptor Activation.

The activity of a compound to inhibit NK1r activation measured as NK1r-mediated increase in intracellular Ca²⁺ was assessed by the following procedure:

CHO cells stably transfected with NK1r were plated in black-walled/clear-bottomed 96-well plates (Costar 3904) at 3.5×10⁴ cells per well and grown for approximately 24 hours in normal growth media in a 37° C. CO₂-incubator.

Before the FLIPR assay the cells of each 96-well plate were loaded with the Ca²⁺-sensitive dye Fluo-3 (TEFLABS 0116) at 4 μM in a loading media consisting of Nut Mix F12 (HAM) with Glutamax I, 22 mM HEPES, 2.5 mM Probenicid (Sigma P-8761) and 0.04% Pluronic F-127 (Sigma P-2443) and kept in the dark for 1 hour in a 37° C. CO₂-incubator. The cells were then washed three times in assay buffer (Hanks balanced salt solution (HBSS) containing 20 mM HEPES, 2.5 mM Probenicid and 0.1% BSA) using a multi-channel pipette leaving them in 150 μl at the end of the last wash. Serial dilutions of a test compound in assay buffer (final DMSO concentration kept below 1%) were automatically pipetted by FLIPR (Fluorometric Imaging Plate Reader) into each test well and the fluorescence intensity was recorded (excitation 488 nm and emission 530 nm) by the FLIPR CCD camera for a 2-min pre-incubation period. 50 μl of the Substance P (NK₁ specific), NKA (NK₂ specific), or Pro-7-NKB (NK₃ specific) agonist solution (final concentration equivalent to an approximate EC₆₀ concentration) was then added by FLIPR into each well already containing 200 μl assay buffer (containing the test compound or vehicle) and the fluorescence was continuously monitored for another 2 min. The response was measured as the peak relative fluorescence after agonist addition and IC₅₀s were calculated from ten-point concentration-response curves for each compound. The IC₅₀s were then converted to pK_{B} values with the following formula:
K_{B}=IC₅₀/1+(EC₆₀ conc. of agonist used in assay/EC₅₀ agonist)
pK_{B}=−log K_{B}
Binding Assay for NK1 Receptors

Membranes were prepared from CHO cells stably transfected with NK1r according to the following method. Cells were detached with Accutase® solution, harvested in PBS containing 5% FBS by centrifugation, washed twice in PBS and resuspended to a concentration of 1×10⁸ cells/ml in Tris-HCl 50 mM, KCl 300 mM, EDTA-N₂ 10 mM pH 7.4 (4° C.). Cell suspensions were homogenized with a UltraTurrax 30 s 12.000 rpm. The homogenates were centrifuged at 38,000×g (4° C.) and the pellet resuspended in Tris-HCl 50 mM pH 7.4. The homogenization was repeated once and the homogenates were incubated on ice for 45 min. The homogenates were again centrifuged as described above and resuspended in Tris-HCl 50 mM pH 7.4. This centrifugation step was repeated 3 times in total. After the last centrifugation step the pellet was resuspended in Tris-HCl 50 mM and homogenized with Dual Potter, 10 strokes to a homogenous solution, and aliquots were removed for protein determination. Membranes were aliquoted and frozen at −80° C. until use.

The radioligand binding assay was performed at room temperature in 96-well microtiter plates (No-binding Surface Plates, Corning 3600) with a final assay volume of 200 μl/well in incubation buffer (50 mM Tris buffer (pH 7.4 RT) containing 0.1% BSA, 40 mg/L Bacitracin, complete EDTA-free protease inhibitor cocktail tablets 20 pills/L (Roche) and 3 mM MnCl₂).

Competition binding curves were done by adding increasing amounts of the test compound. Test compounds were dissolved and serially diluted in DMSO, final DMSO concentration 1.5% in the assay. 50 μl Non-labelled ZD 6021 (a non selective NK-antagonist, 10 μM final conc.) was added for measurement of nonspecific binding. For total binding, 50 μl of 1.5% DMSO (final conc.) in incubation buffer was used. The radiolabeled SP-analogue [³H-Sar,Met(O₂)-Substance P] (4 nM final conc.) was used in binding experiments on hNK1r. For displacement experiments 50 μl radioligand, 3 μl test compound diluted in DMSO and 47 μl incubation buffer were mixed with 5-10 μg cell membranes in 100 μl incubation buffer and incubated for 30 min at room temperature on a microplate shaker.

The membranes were then collected by rapid filtration on Filtermat B(Wallac), presoaked in 0.1% BSA and 0.3% Polyethyleneimine (Sigma P-3143), using a Micro 96 Harvester (Skatron Instruments, Norway). Filters were washed by the harvester with ice-cold wash buffer (50 mM Tris-HCl, pH 7.4 at 4° C., containing 3 mM MnCl₂) and dried at 50° C. for 30-60 min. Meltilex scintillator sheets were melted on to filters using a Microsealer (Wallac, Finland) and the filters were counted in a β-Liquid Scintillation Counter (1450 Microbeta, Wallac, Finland).

Calculation of Binding Affinity (Ki)

The Kᵢ value for the unlabeled ligand was calculated using the Cheng-Prusoff equation (Biochem. Pharmacol. 22:3099-3108, 1973), where L is the concentration of the radioactive ligand used and K_{d} is the affinity of the radioactive ligand for the receptor, determined by saturation binding.

Data was fitted to a four parameter equation using Excel Fit.
Kᵢ=IC₅₀/(1+(L/K_{d}))
Gerbil Foot Tap (NK1r-Specific Test Model)

Male Mongolian gerbils (60-80 g) were purchased from Charles River, Germany. On arrival, they were housed in groups of ten, with food and water ad libitum in temperature- and humidity-controlled holding rooms. The animals were allowed at least 7 days to acclimatize to the housing conditions before experiments. Each animal was used only once and killed immediately after the experiment by heart punctuation or a lethal overdose of pentobarbital sodium.

Gerbils were anaesthetized with isoflurane. Potential CNS-permeable NK1 receptor antagonists were administered intraperitoneally, intravenously or subcutaneously. The compounds were given at various time points (typically 30-120 minutes) prior to stimulation with agonist.

The gerbils were lightly anaesthetized using isoflurane and a small incision was made in the skin over bregma. 10 pmol of ASMSP, a selective NK1receptor agonist, was administered icv in a volume of 5 μl using a Hamilton syringe with a needle 4 mm long. The wound was clamped shut and the animal was placed in a small plastic cage and allowed to wake up. The cage was placed on a piece of plastic tubing filled with water and connected to a computer via a pressure transducer. The number of hind feet taps was recorded.

Radioligand Binding Studies

In saturation experiments using an analogue of Substance P (3H-SarMet-SP) (Drapeau et al., Neuropeptides 10: 43-54, 1987), about similar Kd-values, 2.2 and 2.7 nM and Bmax-values, 4817 and 5285 fmols/mgP, were found in human and gerbil NK1r (NK1-CHO), respectively.

In respect to the similar Kd-values for SarMet-SP found in human and gerbil the results indicate, firstly, that the NK1r present in the two species are of the same kind and, secondly, that probably the binding sites for Substance P are quite similar. From those assumptions it is quite possible that the antagonist binding site is also covered by the peptide. That was also shown when testing different antagonists in cell membranes, from each receptor (Table 4) where similar affinity values (Ki) were obtained with four structurally different NK antagonists.

The affinities of four NKr antagonists were evaluated, ZD 6021 (NKr-unselective), CP 99994 (NK1r-selective), MK 869 (NK1r-selective) and CP 122721 (NK1r-selective). All compounds showed approximately the same affinity to human and gerbil NK1r (Table 4).

In rat NK1r several compounds show markedly lower (>2 orders of magnitude) affinity than in human. We now believe that the difference in affinity may be related to the amino acid sequence difference in human/rat at three positions in the NK1r: Val116/Leu116 and Ile290/Ser290 (Sachais et al., JBC 268:2319-2323, 1993) and Ser80/Cys80 (Fong et al., JBC 267:25668-25671, 1992, Rosenkilde et al., JBC 269:28169-28164, 1994) all located at positions for antagonist binding. Further studies in our own laboratory have shown that mutations of the gerbil NK1r at positions 116 and 290 to the respective rat NK1r markedly reduce the potency of the human-preferable NK1r antagonists. Comparing the human and gerbil NK1receptor, similar amino acids are present at these positions.

In Table 4, Ki values for the tested compound are shown and all compounds showed almost similar Ki values on NK1r in both human and gerbil receptors.

TABLE 4
Affinity (Ki-values) of unselective and selective NKr antagonists to
human and gerbil NK1r

| | Human NK1 | Gerbil NK1 |
|---|---|---|
| Compound | Ki (nM) | Ki (nM) |
| ZD 6021 | 0.31 | 0.38 |
| CP 99994 | 0.33 | 0.81 |
| MK 869 | 0.4 | 0.16 |
| CP 122721 | 0.09 | 0.24 |

Determination of Functional Potency of NK Antagonists

The antagonistic effects of the compounds listed in Table 4 were tested for their functional potency (pKB). Table 5 shows that the human and gerbil potencies were very similar, whereas the rat potencies for all substances were from 1 to 3 orders of magnitude lower.

TABLE 5
Potency (pKB ± SD) of NK antagonists in human, gerbil and rat
NKr. The data shown are inhibitory potency of antagonists following
activation of intracellular Ca2+ mobilization by Substance P
(NK1r) and Neurokinin A (NK2r). Values were obtained from
inhibitory responses of a submaximal (60%) concentration of
Substance P, and the KB-values were calculated from the
Cheng-Prusoff equation and transformed to pKB-values. The in vivo
(GFT) apparent pKB (pKBapp) was determined by estimating the brain
homogenate concentration of antagonist at 50% inhibition. This value
was used in the Cheng-Prusoff equation, as above.

| | human NK1r | gerbil NK1r | gerbil NK1r | rat NK1r |
|---|---|---|---|---|
| Compound | pKB | pKB | pKBapp. | pKB |
| ZD 6021 | 8.53 ± 0.41 | 9.00 ± 0.25 | 8.5 | <6 |
| CP99994 | 8.68 ± 0.25 | 8.94 ± 0.25 | 8.1 | 5.91 ± 0.21 |
| MK 869 | 8.70 ± 0.22 | 8.81 ± 0.15 | 8.9 | 7.26 ± 0.13 |
| CP 122721 | 9.43 ± 0.32 | 9.30 ± 0.05 | 9.9 | 7.10 ± 0.34 |

Inhibitory Effect of NKr Antagonists on ASMSP-Induced GFT

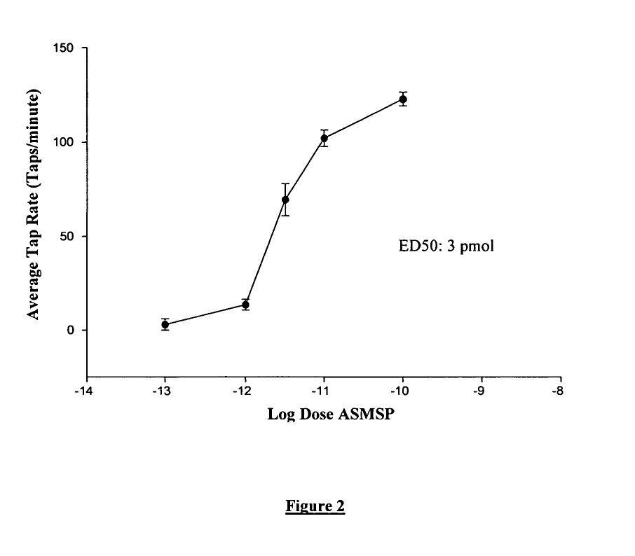

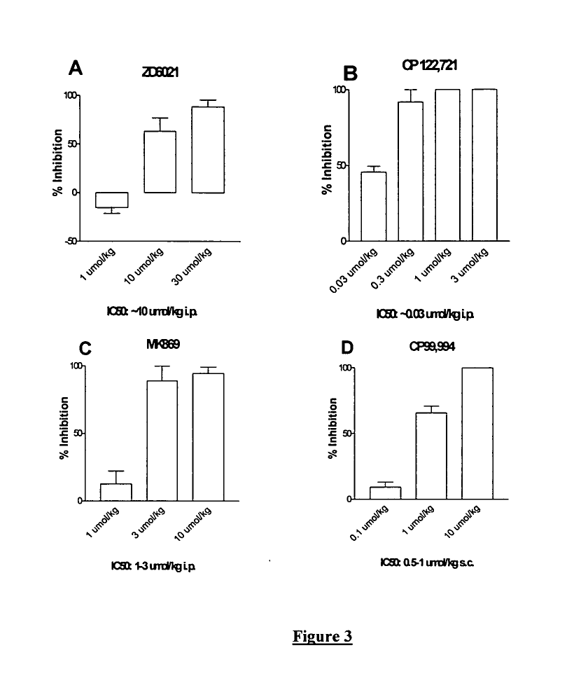

The selective NK1r agonists GR73632 and ASMSP produced dose-dependent stimulation of the foot tapping response with ED₅₀ values of 0.8 and 2.7 pmol respectively (FIG. 2). Both peptides achieved the same efficacy. A dose of 10 pmol ASMSP (˜ED₈₀ dose) was chosen for subsequent antagonist experiments. Four NK1r antagonists were tested. ZD 6021 (Rumsey et al., JPET 298:307-315, 2001) is an unselective NKr antagonist while CP122,721 (McLean et al., JBC 277:900-908, 1996), MK869 (Ballard et al., European Journal of Pharmacology. 412(3):255-64, 2001) and CP99,994 (McLean et al., JPET 267(1):472-9, 1993) are more selective NK1r antagonists. All four NKr antagonists tested produced dose-dependent inhibition of ASMSP-induced foot tapping (FIG. 3). The IC₅₀ values for each compound are shown in the figure. The tentative rank order of potency was CP122721>CP99994>MK869>ZD6021. However, the potency of CP99994 is difficult to compare with the other antagonists since the compound was administered subcutaneously instead of intraperitoneally.

We claim:

1. An isolated polynucleotide molecule comprising a nucleic acid sequence which encodes a gNK1r polypeptide

2. An isolated polynucleotide molecule comprising a nucleic acid sequence which encodes a polypeptide having at least 98% sequence identity to the amino acid sequence disclosed in SEQ ID NO: 2.

3. The isolated polynucleotide according to claim 1, which sequence comprises the sequence set out in SEQ ID NO: 1.

4. The isolated polynucleotide according to claim 2, which sequence comprises the sequence set out in SEQ ID NO: 1.

5. An expression vector comprising a polynucleotide molecule according to any one of claims 1-4.

6. A host cell transformed or transfected with the expression vector according to claim 5.

7. The host cell according to claim 6, which is a mammalian cell.

8. A method for producing a polypeptide, which method comprises culturing a host cell comprising a polynucleotide as claimed in any one of claims 1 to 4, under conditions suitable for the expression of said polypeptide.

9. An isolated or purified polypeptide comprising the gNK1r amino acid sequence set out in SEQ ID NO: 2 or a variant of SEQ ID NO: 2 having at least 98% identity thereto, or an N-terminal or C-terminal fragment thereof of at least 350 amino acids in length.

10. An isolated gNK1r polypeptide selected from: SEQ ID NO: 2 or a fragment thereof selected from the group consisting of SEQ ID NO: 2 positions: 1-377, 1-387, 1-400, 1-406, 10-407, 15-407, 20-407, 25-407, 35-407, 40-390, 15-385, 20-380, 25-370, 30-370 and 25-400.

11. The variant polypeptide according to claim 9, which variant possesses, relative to the position in SEQ ID NO:2, a serine at position 80, a valine at position 1 16 and an isoleucine at position 290.

12. A method for identifying a chemical compound capable of modulating the activity of gNK1r which method comprises:
(i) contacting a chemical compound with a gNK1r polypeptide according to claim 9; and
(ii) measuring an effect of the chemical compound on the activity of the gNK1r polypeptide.

13. A method for identifying a therapeutic agent capable of modulating the activity of gNK1r, which method comprises:
(i) contacting a candidate compound modulator with a gNK1r polypeptide comprising the amino acid sequence set out in SEQ ID NO: 2 or a variant of SEQ ID NO: 2 having at least 98% sequence identity thereto, or an N-terminal or C-terminal fragment thereof of at least 377 amino acids in length; and
(ii) measuring an effect of the candidate compound modulator on the activity of the gNK1r polypeptide.

14. A method for identifying a therapeutic agent capable of modulating the activity of gNK1r, which method comprises:
(i) contacting a candidate compound modulator with a host cell which expresses a gNK1r polypeptide comprising the amino acid sequence set out in SEQ ID NO: 2 or a variant of SEQ ID NO: 2 having at least 98% identity thereto, or a biologically active fragment thereof; and
(ii) measuring an effect of the candidate compound modulator on the activity of gNK1r.

15. A method of making a pharmaceutical composition which comprises:
(i) identifying a chemical compound capable of modulating the activity of gNK1r; and
(ii) mixing the compound thus identified with a pharmaceutically acceptable diluent or carrier.

16. A pharmaceutical composition which comprises a gNK1r modulator, or a pharmaceutically acceptable salt thereof, in association with a pharmaceutically acceptable diluent or carrier.


SEQUENCE LISTING (22 sequences)

SEQ ID NO: 1 - DNA, 1224 bp, Meriones unguiculatus
atggataacgtcctccctggggactcggacctcttccccaacatctccaccaacagttccgagtccaaccaattcgtacagcctgcctggcaaattgtcctttgggcagctgcctacacggtcatcgtggtgacctccgtggtgggcaacgtggtggtgatgtggatcattttggcccacaagagaatgaggacagtgaccaattacttcctggtgaacctggccttcgctgaggcctccatggccgcattcaacacggtggtgaacttcacctacgccgtccacaacgagtggtactacggcctcttctactgcaagttccacaacttcttccccattgctgctgtcttcgccagcatctactccatgacagcagtggccttcgacaggtacatggccatcatccaccctctccagccccggctgtcggccaccgccaccaaggtggtcatctttgtcatctgggtgctggctctcctgttggcctttccgcagggctactactccaccacggagaccatgcccggcagagtagtgtgcatgatcgagtggccggaacaccccaacaggacttacgagaaagcgtaccacatctgtgtgacggtgctgatctacttcctgcccctgctggtgattggctacgcctacactgtcgtaggaatcacactgtgggccagcgagatccccggggactcctccgaccgctaccacgagcaagtctccgccaagcgcaaggtggtcaaaatgatgatcgtggtcgtgtgcaccttcgccatctgctggctgcccttccacgtcttcttcctcctgccctacatcaacccggacctctacgttaaaaagttcatccagcaggtctacctggccatcatgtggctagccatgagctccaccatgtacaaccccatcatctactgctgcctcaatgacaggttccgtctgggcttcaaacacgcttttcgctgctgtcccttcatcagtgctggtgattatgaggggctggaaatgaaatccacccgatacctccagacccagggcagtgtctacaaggtcagccgcctggagaccaccatctccactgtggtgggagcccatgaagatgaggcagaagaaggccccaaggccacaccttcatccttggatctcacctccaatggctcttctcgtagcaactcgaagaccatgacagaaagctccagcttctactctaacatgctggcctag

SEQ ID NO: 2 - protein, 407 aa, Meriones unguiculatus
MDNVLPGDSDLFPNISTNSSESNQFVQPAWQIVLWAAAYTVIVVTSVVGNVVVMWIILAHKRMRTVTNYFLVNLAFAEASMAAFNTVVNFTYAVHNEWYYGLFYCKFHNFFPIAAVFASIYSMTAVAFDRYMAIIHPLQPRLSATATKVVIFVIWVLALLLAFPQGYYSTTETMPGRVVCMIEWPEHPNRTYEKAYHICVTVLIYFLPLLVIGYAYTVVGITLWASEIPGDSSDRYHEQVSAKRKVVKMMIVVVCTFAICWLPFHVFFLLPYINPDLYVKKFIQQVYLAIMWLAMSSTMYNPIIYCCLNDRFRLGFKHAFRCCPFISAGDYEGLEMKSTRYLQTQGSVYKVSRLETTISTVVGAHEDEAEEGPKATPSSLDLTSNGSSRSNSKTMTESSSFYSNMLA

SEQ ID NO: 3 - DNA, 28 bp, Artificial
  Polynucleotide for use in polymerase chain reaction
gctgcccttccacatcttcttcctcctg

SEQ ID NO: 4 - DNA, 27 bp, Artificial
  Polynucleotide for use in polymerase chain reaction
gccagcaggagagccaggacccagatg

SEQ ID NO: 5 - DNA, 19 bp, Artificial
  Polynucleotide for use in polymerase chain reaction
aggcatctgcaacaaggtc

SEQ ID NO: 6 - DNA, 22 bp, Artificial
  Polynucleotide for use in polymerase chain reaction
aaccattatgaccctttccaga

SEQ ID NO: 7 - DNA, 32 bp, Artificial
  Polynucleotide for use in polymerase chain reaction
ggatccgccaccatggataacgtcctccctgg

SEQ ID NO: 8 - DNA, 29 bp, Artificial
  Polynucleotide for use in polymerase chain reaction
gatatcatgcccttgaaatatgcccactg

SEQ ID NO: 9 - protein, 23 aa, Meriones unguiculatus and Homo sapiens
IVLWAAAYTVIVVTSVVGNVVVM

SEQ ID NO: 10 - protein, 23 aa, Rattus norvegicus
IVLWAAAYTVIVVTSVVGNVVVI

SEQ ID NO: 11 - protein, 22 aa, Meriones unguiculatus and Homo sapiens
TVTNYFLVNLAFAEASMAAFNT

SEQ ID NO: 12 - protein, 22 aa, Rattus norvegicus
TVTNYFLVNLAFAEACMAAFNT

SEQ ID NO: 13 - protein, 22 aa, Meriones unguiculatus and Homo sapiens
FHNFFPIAAVFASIYSMTAVAF

SEQ ID NO: 14 - protein, 22 aa, Rattus norvegicus
FHNFFPIAALFASIYSMTAVAF

SEQ ID NO: 15 - protein, 20 aa, Meriones unguiculatus and Rattus norvegicus
VIFVIWVLALLLAFPQGYYS

SEQ ID NO: 16 - protein, 20 aa, Homo sapiens
VICVIWVLALLLAFPQGYYS

SEQ ID NO: 17 - protein, 25 aa, Meriones unguiculatus and Rattus norvegicus
AYHICVTVLIYFLPLLVIGYAYTVV

SEQ ID NO: 18 - protein, 25 aa, Homo sapiens
VYHICVTVLIYFLPLLVIGYAYTVV

SEQ ID NO: 19 - protein, 22 aa, Meriones unguiculatus and Rattus norvegicus
MMIVVVCTFAICWLPFHVFFLL

SEQ ID NO: 20 - protein, 22 aa, Homo sapiens
MMIVVVCTFAICWLPFHIFFLL

SEQ ID NO: 21 - protein, 25 aa, Meriones unguiculatus and Homo sapiens
QQVYLAIMWLAMSSTMYNPIIYCCL

SEQ ID NO: 22 - protein, 25 aa, Rattus norvegicus
QQVYLASMWLAMSSTMYNPIIYCCL